(19) United States
(12) Patent Application Publication
(10) Pub. No.: US 2011/0288327 A1
(43) Pub. Date: Nov. 24, 2011

(54) PROCESS FOR THE MANUFACTURE OF COMPOUNDS COMPRISING NITRILE FUNCTIONAL GROUPS

(75) Inventor: Sergio Mastroianni, Lyon (FR)

(73) Assignee: RHODIA OPERATIONS, Aubervilliers (FR)

(21) Appl. No.: 13/146,610

(22) PCT Filed: Jan. 18, 2010
(86) PCT No.: PCT/EP2010/050521

§ 371 Date: Aug. 10, 2011

(30) Foreign Application Priority Data
Jan. 29, 2009 (FR) 0950559

(51) Int. Cl. C07C 253/10

(52) U.S. Cl. 558/338

(57) ABSTRACT

A method for producing compounds including at least one nitrile function by the hydrocyanation of a compound including at least one non-conjugated unsaturation is described. A method for producing compounds including at least one nitrile function by the hydrocyanation of an organic compound including at least one non-conjugated unsaturation including 2 to 20 carbon atoms by reacting with hydrogen cyanide in the presence of a catalytic system including at least one nickel complex in a zero oxidation state with at least one organophosphorus ligand selected from the group including organophosphites, organophosphonites, organophosphinites and organosphosphines and a co-catalyst such as a Lewis acid consisting of a mixture of Lewis acids is also described.

The present invention relates to a process for the manufacture of compounds comprising at least one nitrile functional group by hydrocyanation of a compound comprising at least one unconjugated unsaturation.

It relates more particularly to a manufacturing process employing the reaction of hydrogen cyanide with an organic compound comprising an unconjugated unsaturation in the presence of a catalytic system comprising a complex of nickel in the zero oxidation state (hereinafter referred to as Ni(0)) with at least one organophosphorus ligand and a cocatalyst belonging to the family of Lewis acids.

Such processes have been known for many years and are made use of industrially, in particular for the production of a major chemical intermediate, adiponitrile. This intermediate is used in particular in the manufacture of hexamethylenediamine, which is an important monomer in the manufacture of polyamides and also an intermediate in the synthesis of diisocyanate compounds.

Thus, Du Pont de Nemours has developed and made use of a process for the manufacture of adiponitrile by double hydrocyanation of butadiene. This reaction is generally catalyzed by a catalytic system comprising a complex of Ni(0) with organophosphorus ligands. This system also comprises a cocatalyst, in particular in the second hydrocyanation stage, that is to say stage of hydrocyanation of unsaturated compounds comprising a nitrile functional group, such as pentenenitriles, to give dinitrile compounds.

Many cocatalysts have been provided in the patents and are generally compounds belonging to the family of Lewis acids. One of the roles of this cocatalyst or promoter is to limit the production of byproducts and thus to promote the formation of linear dinitrile compounds in comparison with the formation of branched dinitriles.

Thus, many metal halides, such as zinc chloride, zinc bromide, stannous chloride or stannous bromide have already been provided, for example, in U.S. Pat. No. 3,496,217. Zinc chloride is the preferred cocatalyst.

Organic boron compounds, such as triphenylboron or compounds comprising two boron atoms, such as are described in U.S. Pat. No. 3,864,380 and U.S. Pat. No. 3,496,218, and also organic tin compounds, as in U.S. Pat. No. 4,874,884, have also been provided.

Cocatalysts comprising several acid sites, in particular two acid sites have been provided in French Patent Applications Nos 08 00381 and 08 05821, which have not yet been published.

These cocatalysts have different properties and make it possible to obtain different selectivities for linear dinitriles, such as adiponitrile. Some of these cocatalysts exhibit disadvantages related to the difficulty in extracting them from the reaction medium or to the possibility and ease of extracting the catalytic system or the ligand of the Ni(0) in the presence of this cocatalyst in order to recycle it.

Provision has also been made to use, as cocatalyst, a mixture of Lewis acids, in particular when one of the cocatalysts is triphenylboron, as is described in U.S. Pat. No. 4,874,884, or to combine, with a Lewis acid, another compound composed of an aluminium or titanium alkoxide, as described in Patent application WO2004/087314.

There still exists a need to find novel catalytic systems which make it possible to obtain selectivities for linear dinitriles of acceptable levels and which are easy to use and/or to improve the kinetics and the dinitriles yield of the hydrocyanation reaction.

One of the aims of the present invention is to provide a novel catalytic system which comprises a novel combination of compatible specific cocatalysts and which gives suitable levels of selectivity for adiponitrile and suitable levels of yield of dinitriles in the reaction for the hydrocyanation of pentenitriles.

To this end, the invention provides a process for the manufacture of compounds comprising at least one nitrile functional group by hydrocyanation of an organic compound comprising at least one unconjugated unsaturation comprising from 2 to 20 carbon atoms by reaction with hydrogen cyanide in the presence of a catalytic system comprising a complex of nickel in the zero oxidation state with at least one organophosphorus ligand chosen from the group consisting of organophosphites, organophosphonites, organophosphinites and organophosphines and a cocatalyst, characterized in that the cocatalyst is composed of a mixture of at least two Lewis acids, at least one of which is an organometallic compound corresponding to the following general formula I:

[(R)_{a—}(X)_{y}-]ₙM-(O)ₚ-M₁[-(X)_{z—}(R₁)ₐ₁]ₙ₁

in which:
M and M₁, which are identical or different, represent an element chosen from the group consisting of the following elements: B, Si, Ge, Sn, Pb, Mo, Ni, Fe, W, Cr, Zn, Al, Cd, Ga and In,
R and R₁, which are identical or different, represent an aliphatic radical or a radical comprising an aromatic or cycloaliphatic ring, which is or is not substituted and which may or may not be bridged, or a halide radical,
X represents an oxygen, nitrogen, sulphur or silicon atom,
y, z and p are identical or different integers equal to 0 or 1,
n and n₁ are integers equal to the valency, reduced by 1, of the elements M and M₁,
a and a1 are identical or different integers equal to the valency, reduced by 1, of the element X if y and z are equal to 1, or equal to 1 if y and z are equal to 0.

Advantageously, R and R1, which are identical or different, represent an aromatic, aliphatic or cycloaliphatic radical, which is or is not substituted and which may or may not be bridged, or a halide radical.

In the above formula, when p is equal to 0, the bond between the elements M and M₁ is a single or multiple covalent bond, depending on the nature of the elements M and M₁.

In the above formula, a is equal to the valency, reduced by 1, of the element X if y is equal to 1 and a is equal to 1 if y is equal to 0. Likewise, a1 is equal to the valency, reduced by 1, of the element X if z is equal to 1 and a1 is equal to 1 if z is equal to O.

According to a preferred characteristic of the invention, the organometallic compound of formula I is advantageously chosen from the group of the following compounds:
bis(neopentyl glycolato)diboron (RN CAS 201733-56-4)
bis(hexylene glycolato)diboron (RN CAS 230299-21-5)
bis(pinacolato)diboron (RN CAS 73183-34-3)
tetrakis(pyrrolidino)diborane (RN CAS 158752-98-8)
hexamethyldisilane (RN CAS 1450-14-2)
tetraphenyldimethyldisilane (RN CAS 1172-76-5)
diphenyltetramethyldisilane (RN CAS 1145-98-8)
tris(trimethylsilyl)silane (RN CAS 1873-77-4)
tetrakis(trimethylsilyl)silane (RN CAS 4098-98-0)
hexaphenyldisilane (RN CAS 1450-23-3)
hexamethyldigermane (RN CAS 993-52-2)
hexaethyldigermane (RN CAS 993-62-4)
hexaphenyldigermane (RN CAS 2816-39-9)
hexamethylditin (RN CAS 661-69-8)
hexabutylditin (RN CAS 813-19-4)
hexaphenylditin (RN CAS 1064-10-4)
triphenylstannyldimethylphenylsilane (RN CAS 210362-76-8)
triphenylgermanium; triphenyltin (RN CAS 13904-13-7)
hexaphenyldilead (RN CAS 3124-01-4)
cyclopentadienyliron dicarbonyl dimer (RN CAS 38117-54-3)
cyclopentadienyl chromium dicarbonyl dimer (RN CAS 37299-12-0)
cyclopentadienylnickel carbonyl dimer (RN CAS 12170-92-2)
cyclopentadienyltungsten tricarbonyl dimer (RN CAS 12566-66-4)
methylcyclopentadienylmolybdenum tricarbonyl dimer (RN CAS 33056-03-0) and the compounds with the following formulae:

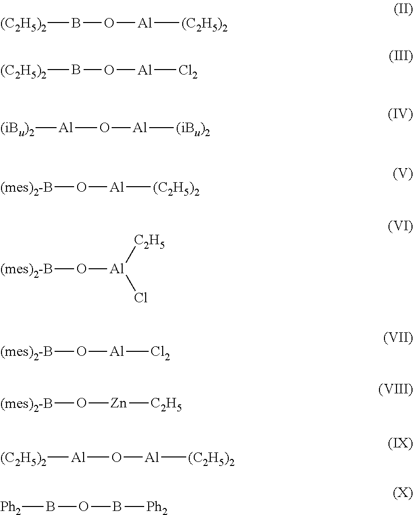

in which
iBu represents the isobutyl radical
mes represents a mesityl (2,4,6 trimethylphenyl) group, and
Ph represents a phenyl group.
The compound of formula (IV) is illustrated under the No. CAS 998-00-5 and is referred to as TIBAO in the continuation of the present text.
The compound of formula (X) is listed under the No. CAS 4426-21-5.

These compounds are described in the literature, along with their process of manufacture. The RN CAS registry number is given solely by way of information. The majority of these compounds are available commercially.

According to the invention, the Lewis acid which is present in combination with the compound of formula I can be chosen from the various and numerous Lewis acids already described and used in catalytic systems for the hydrocyanation reaction, in particular the reaction for the hydrocyanation of pentenenitriles. Such Lewis acids are described in the patents mentioned above in describing the state of the art.

Mention may be made, as nonlimiting examples of Lewis acids suitable for the catalytic system of the invention, of a large number of compounds comprising metal cations combined with a very large variety of anions. Thus, by way of example, the cations can be zinc, cadmium, beryllium, aluminium, gallium, indium, lead, titanium, vanadium, niobium, scandium, chromium, molybdenum, tungsten, manganese, rhenium, palladium, thorium, erbium, iron and cobalt.

Mention may be made, as preferred anions, of halides, such as fluoride, chloride, bromide and iodide, anions of organic fatty acids comprising 2 to 7 carbon atoms, or the anions HPO₃²⁻, H₂PO₂⁻, CF₃COO⁻, OSO₂C₇F₁₅⁻ or SO₄²⁻.

Other Lewis acids belonging to the family of organic boron or tin compounds, such as triphenylboron, can also be used.

In a preferred form of the invention, the catalytic system of the invention comprises a cocatalyst in accordance with the invention in a molar ratio of cocatalyst, with respect to the number of nickel atoms, of between 0.01 and 50 and preferably between 0.1 and 10. This concentration of cocatalyst corresponds to the total concentration of Lewis acid.

In the catalytic system of the invention, the compound of formula I represents at least 0.1 mol % of the mixture of Lewis acids, advantageously at least 1%, preferably at least 5%, more preferably still at least 10%. In the case where the second Lewis acid does not correspond to the formula I, this second Lewis acid is advantageously present in the mixture at a molar ratio of at least 50%.

According to a preferred characteristic of the invention, the Lewis acid used in combination with the compound of formula I is advantageously chosen from the group of the Lewis acids comprising just one acid site which are listed in U.S. Pat. No. 3,496,217, U.S. Pat. No. 3,864,380, U.S. Pat. No. 3,496,218 and U.S. Pat. No. 4,874,884. Mention may be made, as Lewis acid which is particularly preferred in this list, of zinc chloride and triphenylboron.

The catalytic system of the invention comprises a complex of Ni(0) with at least one organophosphorus compound, preferably a monodentate compound, such as triphenyl phosphite or tritolyl phosphite, for example described in U.S. Pat. No. 3,496,215, DE19953058, FR 1 529 134, FR 2 069 411, U.S. Pat. No. 3,631,191, U.S. Pat. No. 3,766,231 and FR 2 523 974, or a bidentate compound, such as the organophosphite compounds described in Patents WO 9906355, WO 9906356, WO 9906357, WO 9906358, WO 9952632, WO 9965506, WO 9962855, U.S. Pat. No. 5,693,843, WO 961182, WO 9 622 968, U.S. Pat. No. 5,981,772, WO 0136429, WO 9964155, WO 0213964 and U.S. Pat. No. 6,127,567.

It is also possible to use complexes of Ni(0) with monodentate or bidentate organophosphine compounds, such as described in Patents WO 02/30854, WO 02/053527, WO 03/068729, WO 04/007435, WO 04/007432, FR 2 845 379 and WO 2004/060855, and more particularly trithienylphosphine, described in unpublished French Application No. 0803373, and DPPX, described in Patent WO 2003031392.

Likewise, the catalytic system of the invention can comprise a complex of Ni(0) with monodentate or bidentate organophosphorus compounds belonging to the family of the organophosphonites or organophosphinites.

It is also possible to use the cocatalysts of the invention with a complex of Ni(0) obtained with a mixture of monodentate organophosphite ligand and bidentate ligand chosen from the families of compounds belonging to the organophosphites, organophosphonites, organophosphinites or organophosphines, such as described in Patents WO03011457 and WO2004/065352, or mixtures of monodentate ligands, as described in the as yet unpublished French Patent Application No. 08 03374.

The hydrocyanation process is described in several patents, including those mentioned above, and also in the papers by C. A. Tolman published in the reviews Organometallics, 3 (1984) 33, Advances in Catalysis (1985), 33-1, and the Journal of Chemical Education (1986), vol. 63, No. 3, pages 199-201.

Briefly, the process for the manufacture of compounds comprising at least one nitrile functional group and more particularly dinitrile compounds, such as adiponitrile, consists in reacting, in a first stage, a diolefin, such as 1, 3-butadiene, with hydrogen cyanide, generally in the absence of solvent and in the presence of a catalytic system. The reaction is carried out under pressure in order to be in a liquid medium. The unsaturated nitrile compounds are separated by successive distillations. The linear nitrile compounds, such as pentenenitriles, are fed to a second hydrocyanation stage.

Advantageously, the nonlinear unsaturated nitriles obtained in the first stage are subjected to an isomerization stage in order to convert them to linear unsaturated nitriles, which are also introduced into the second hydrocyanation stage.

In the second hydrocyanation stage, the linear unsaturated nitriles are reacted with hydrogen cyanide in the presence of a catalytic system.

The dinitrile compounds formed are separated by successive distillations after extraction of the catalytic system from the reaction medium. Several processes for the extraction of the catalytic system are described, for example, in U.S. Pat. Nos. 3,773,809, 4,082,811, 4,339,395 and 5,847,191. Generally, the catalytic system can be separated from the reaction medium by settling into two phases obtained by the control of the ratios of mononitrile compounds to the dinitrile compounds present in the medium. This separation can be improved by the addition of ammonia. It is also possible to precipitate the catalytic system in order to recover it and recycle it or to use a nonpolar solvent in order to extract the catalytic system and to separate it from the nitrile products.

The temperature conditions for these different stages are between 10 and 200° C.

The catalytic systems used in the first and second hydrocyanation stages and in the isomerization stage are generally similar, that is to say that they comprise an identical Ni(0) complex. However, the ratio of the number of nickel atoms to the number of ligand molecules can be different in each of these stages, and also the concentration of the catalytic system in the medium.

Preferably, the cocatalyst is present solely in the catalytic system used for the second hydrocyanation stage. However, it can also be present in the isomerization stage and optionally in the first stage.

The characteristics and performances of the process and thus of the catalytic system used are determined and illustrated by the yield of dinitrile compounds (RR)_{DN} and by the linearity (L) of linear dinitriles produced, that is to say the number of moles of linear dinitriles with respect to the number of moles of dinitriles formed. In the case of the manufacture of adiponitrile, the linearity corresponds to the percentage of moles of adiponitrile (AdN) obtained with respect to the number of moles of dinitriles formed (AdN+ESN+MGN).

The use of this specific combination of Lewis acids as catalyst makes it possible to improve the kinetics of the invention and the catalytic performances. Thus, it is possible with the catalytic system of the invention to reduce the concentration of catalyst without affecting the productive output of the reaction.

A better illustration of the invention will be obtained from the examples given below, solely by way of indication, relative to the manufacture of adiponitrile by hydrocyanation of 3-pentenenitrile. In these examples, the 3-pentenenitrile used is a compound sold by Aldrich.

Abbreviations Used in the Examples Have the Following Meanings:
cod: cyclooctadiene
3PN: 3-pentenenitrile
AdN: adiponitrile
ESN: ethylsuccinonitrile
MGN: methylglutaronitrile
LA: Lewis acid
DN: dinitriles (AdN, MGN or ESN)
TTP: tri(para-tolyl) phosphite
TIBAO: tetraisobutyldialuminoxane
BPDB: bis(pinacolato)diboron
DPPX: 1,2-bis(diphenylphosphinomethyl)benzene
Linearity (L): ratio of the number of moles of AdN formed to the number of moles of dinitriles formed (sum of the moles of AdN, ESN and MGN)
RY_{(DN)}: yield of dinitriles corresponding to the ratio of the number of moles of dinitriles formed to the number of moles of 3PN charged

The compounds 3PN, Ni(cod)₂, TTP, ZnCl₂, TIBAO, diphenylborinic anhydride (Ph₂BOBPh₂), DPPX, trithienylphosphine and BPDB are available commercially.

### EXAMPLES 1 to 9

### Hydrocyanation of 3-PN to Give AdN With Just One Lewis Acid (Comparative Examples)

The procedure employed to carry out these examples is described below:

The following are successively charged, under an argon atmosphere, to a 60 ml glass tube of Schott type equipped with a septum stopper:
the ligand:
   for the monodentate ligands (TTP or trithienylphosphine): 5 equivalents (5 mol of ligands per one mol of nickel)
   for the bidentate ligands (DPPX): 2.5 equivalents (2.5 mol of ligands per one mol of nickel)
1.21 g (15.mmol, 30 equivalents) of anhydrous 3PN
138 mg (0.5 mmol, 1 equivalent) of Ni(cod)₂
LA: the natures and the amounts of the Lewis acids are shown in Table I below:

The mixture is brought with stirring to 70° C. Acetone cyanohydrin is injected into the reaction medium via a syringe driver at a flow rate of 0.45 ml per hour. After injecting for 3 hours, the syringe driver is halted. The mixture is cooled to ambient temperature, diluted with acetone and analyzed by gas chromatography.

The results are combined in the following Table I.

In the examples, the total amount of Lewis acid added is determined in order to obtain a ratio of active site of the Lewis acids with respect to a nickel atom equal to 1.

TABLE I

| | | | LA/Ni | Linearity | |
|---|---|---|---|---|---|
| Example | Ligand | LA | (molar) | (L) | RY_{(DN)} |
| 1 | TTP | ZnCl₂ | 1 | 82.4 | 58.5 |
| 2 | TTP | TIBAO | 0.5 | 76.8 | 31.3 |
| 3 | TTP | Ph₂BOBPh₂ | 0.5 | 73.8 | 1.2 |
| 4 | Trithienylphosphine | TIBAO | 0.5 | 64.1 | 70.6 |
| 5 | Trithienylphosphine | Ph₂BOBPh₂ | 0.5 | 84.7 | 32.3 |
| 6 | DPPX | TIBAO | 0.5 | 84.7 | 65.3 |
| 7 | DPPX | Ph₂BOBPh₂ | 0.5 | 89.1 | 19.2 |
| 8 | DPPX | ZnCl₂ | 1 | 59 | 44.3 |
| 9 | DPPX | BPDB | 0.5 | 93.7 | 4.6 |

### EXAMPLES 10 to 16

### Hydrocyanation of 3-PN to Give AdN With a Mixture of Lewis Acids (Examples in Accordance With the Invention)

In the following examples, the ratio [(total number of acid sites per molecule of LA1)+(total number of acid sites per molecule of LA2)] with respect to a nickel atom is set at 1.

The procedure used is identical to that described in Comparative Examples 1 to 9.

The results obtained and the natures of the ligands and Lewis acids are listed in Table II below:

TABLE II

| | | | LA1/Ni | | LA2/Ni | Linearity | |
|---|---|---|---|---|---|---|---|
| Example | Ligand | LA1 | (molar) | LA2 | (molar) | (L) | RY_{(DN)} |
| 10 | TTP | TIBAO | 0.1 | Ph₂BOBPh₂ | 0.4 | 82.5 | 24.6 |
| 11 | TTP | TIBAO | 0.25 | ZnCl₂ | 0.5 | 82.4 | 67.1 |
| 12 | TTP | TIBAO | 0.1 | ZnCl₂ | 0.8 | 83 | 78.7 |
| 13 | Trithienyl- | TIBAO | 0.1 | Ph₂BOBPh₂ | 0.4 | 80.8 | 43.9 |
| | phosphine | | | | | | |
| 14 | DPPX | TIBAO | 0.25 | Ph₂BOBPh₂ | 0.25 | 88.6 | 57.4 |
| 15 | DPPX | TIBAO | 0.25 | BPDB | 0.25 | 86.5 | 67.0 |
| 16 | DPPX | BPDB | 0.25 | ZnCl₂ | 0.5 | 70.5 | 30.5 |

These results show an increase in the yield of dinitrile compounds RY_{(DN)} while retaining an equivalent linearity (L).

1. A process for manufacturing compounds comprising at least one nitrile functional group, the process comprising hydrocyanation of an organic compound comprising at least one unconjugated unsaturation comprising from 2 to 20 carbon atoms by reaction with hydrogen cyanide in the presence of a catalytic system comprising a complex of nickel in the zero oxidation state with at least one organophosphorus ligand selected from the group consisting of organophosphites, organophosphonites, organophosphinites and organophosphines and a cocatalyst, wherein the cocatalyst is comprised of a mixture of at least two Lewis acids, at least one of which is an organometallic compound corresponding to the general formula I:
[(R)_{a—}(X)_{y}-]ₙM-(O)ₚ-M₁[-(X)_{z—}(R₁)ₐ₁]ₙ₁
in which:
M and M₁, which are identical or different, represent an element selected from the group consisting of: B, Si, Ge, Sn, Pb, Mo, Ni, Fe, W, Cr, Zn, Al, Cd, Ga and In,
R and R₁, which are identical or different, represent an aliphatic radical or a radical comprising an aromatic or cycloaliphatic ring, which is or is not substituted and which is optionally bridged, or a halide radical,
X represents an oxygen, nitrogen, sulphur or silicon atom,
y, z and p are identical or different integers equal to 0 or 1,
n and n₁ are integers equal to the valency, reduced by 1, of the elements M and M₁,
a and a1 are identical or different integers equal to the valency, reduced by 1, of the element X if y and z are equal to 1, or equal to 1 if y and z are equal to 0.

2. The process according to claim 1, wherein R and R₁, which are identical or different, represent an aromatic, aliphatic or cycloaliphatic radical, which is or is not substituted and which is optionally bridged, or a halide radical.

3. The process according to claim 1, wherein the compound of formula I is elected from the group consisting of:
bis(neopentyl glycolato)diboron;
bis(hexylene glycolato)diboron;
bis(pinacolato)diboron;
tetrakis(pyrrolidino)diborane;
hexamethyldisilane;
tetraphenyldimethyldisilane;
diphenyltetramethyldisilane;
tris(trimethylsilyl)silane;
tetrakis(trimethylsilyl)silane;
hexaphenyldisilane;
hexamethyldigermane;
hexaethyldigermane;
hexaphenyldigermane;
hexamethylditin;
hexabutylditin;
hexaphenylditin;
triphenylstannyldimethylphenylsilane;
triphenylgermanium; triphenyltin;
hexaphenyldilead;
cyclopentadienyliron dicarbonyl dimer;
cyclopentadienyl chromium dicarbonyl dimer;
cyclopentadienylnickel carbonyl dimer;
cyclopentadienyltungsten tricarbonyl dimer;
methylcyclopentadienylmolybdenum tricarbonyl dimer; and compounds with the following formulae: 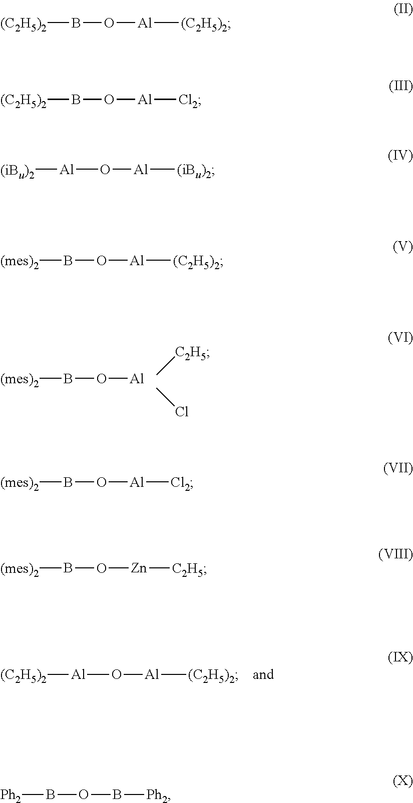
in which
iBu represents the isobutyl radical
mes represents a mesityl (2,4,6 trimethylphenyl) group, and
Ph represents a phenyl group.

4. The process according to claim 1, wherein the catalytic system comprises a molar ratio cocatalyst with respect to the moles of Ni of between 0.1 and 10.

5. The process according to claim 1, wherein the compound of formula I is present in the mixture of Lewis acids at a concentration of at least 0.1 mol %, with respect to the total number of moles of Lewis acids.

6. The process according to claim 5, wherein the compound of formula I is present at at least 5 mol %.

7. The process according to claim 6, wherein the compound of formula I is present at at least 10 mol %.

8. The process according to claim 1, wherein when the mixture of Lewis acids comprises a Lewis acid not corresponding to the formula I, this Lewis acid is present at a molar concentration of at least 50%.

9. The process according to claim 1, wherein the organophosphorus ligand is selected from the group consisting of a monodentate organophosphorus and a bidentate organophosphorus compound.

10. The process according to claim 1, wherein the organic compounds to be converted to dinitrile compounds are pentenenitrile compounds.

11. The process according to claim 10, wherein the compound comprising at least one nitrile functional group is at least one of adiponitrile, methylglutaronitrile and succinonitrile.

12. The process according to claim 5, wherein the compound of formula I is present in the mixture of Lewis acids at a concentration of at least 1 mol %.
